(19) United States
(12) Patent Application Publication
(10) Pub. No.: US 2011/0103543 A1
(43) Pub. Date: May 5, 2011

(54) SCATTER CORRECTION BASED ON RAW DATA IN COMPUTER TOMOGRAPHY

(76) Inventor: Thomas Flohr, Uehlfeld (DE)

(21) Appl. No.: 12/916,712

(22) Filed: Nov. 1, 2010

(30) Foreign Application Priority Data
Nov. 2, 2009 (DE) 10 2009 051 635.2

(51) Int. Cl. A61B 6/03; G06K 9/40

(52) U.S. Cl. 378/4; 382/264

(57) ABSTRACT

A method is disclosed for reconstructing image data of an object under examination from measured data, with the measured data having been detected beforehand during a relative rotational movement between a radiation source of a computer tomography system and the object under examination. In at least one embodiment, a radiation scatter correction variable is determined, which is subjected to a low pass filtering. The filtered radiation scatter correction variable is connected with the measured data to correct the measured data, and image data is reconstructed from the measured data thus corrected.

### PRIORITY STATEMENT

The present application hereby claims priority under 35 U.S.C. §119 on German patent application number DE 10 2009 051 635.2 filed Nov. 2, 2009, the entire contents of which are hereby incorporated herein by reference.

### FIELD

At least one embodiment of the invention generally relates to a method for reconstructing image data of an object under examination from measured data, which was detected beforehand during a relative rotational movement between a radiation source of a computer tomography system and the object under examination.

### BACKGROUND

Methods for scanning an object under examination using a CT system are generally known. Circular scannings, sequential circular scannings with advance or spiral scannings are used here for instance. Other types of scannings, which are not based on circular movements, are also possible, for instance scans with linear segments. Absorption data of the object under examination is received from different recording angles with the aid of at least one x-ray source and at least one opposite detector and this thus collected absorption data and/or projections are calculated to form sectional images through the object under examination by means of corresponding reconstruction methods.

To reconstruct computer tomography images from x-ray CT data records of a computer tomography device (CT device), i.e. from the detected projections, a so-called Filtered Back Projection (FBP) method is nowadays used as a standard method. Following the data acquisition, a so-called ‘rebinning’ step is usually implemented, in which the data generated with the beam propagating from the source in the manner of a fan is rearranged such that it exists in a form which would exist were the detector to be struck by x-rays rays running in parallel onto the detector. The data is then transformed into the frequency range. A filtering takes place in the frequency range, and the filtered data is then back-transformed. With the aid of the thus resorted and filtered data, a back projection to the individual voxels then takes place within the volume of interest.

A problem arising increasingly with an increasing number of detector lines, i.e. with an increasing detector width, is radiation scatter. It is namely possible for an x-ray quantum not to be absorbed by the object under examination, but instead scattered, i.e. deflected in its direction. This means that a specific detector element also measures x-ray quanta which do not originate from the beam connecting the x-ray source with the respective detector element. This effect is referred to as a forward scatter. This results in unwanted artifacts in the reconstructed CT images.

CT devices with two x-ray sources, so-called dual-source devices, also exist. If both x-ray sources are operated with the same x-ray spectrum, this significantly increases the time resolution of the CT images, because the time for the data acquisition halves as a result of the two x-ray sources. This is particularly desirable in the case of moving objects under examination. On the other hand, it is also possible to operate the two x-ray sources with different acceleration voltages and thus different x-ray spectra, so that a dual-energy recording takes place. This allows statements to be made about the composition of the detected tissue.

The presence of radiation scatter is also a known problem in the case of dual-source recordings. In addition to the afore-cited forward scatter, transverse scatter also occurs in the case of dual-source devices. This means that radiation from an x-ray source, which is scattered onto the surface or into the interior of the object under examination, reaches the detector which is not assigned to this x-ray source. This is undesirable since it is only the evaluation of the transmitted radiation of the x-ray source assigned to the respective detector that is of interest.

### SUMMARY

In at least one embodiment of the invention, a method is disclosed for reconstructing CT images, whereby the unwanted effects of the radiation scatter are to be reduced. Furthermore, in at least one embodiment a corresponding control and computing unit, a CT system, a computer program and a computer program product are also disclosed.

At least one embodiment is directed to a method, a control and computing unit, a CT system, a computer program and/or a computer program product. Advantageous embodiments and developments form the subject matter of the subclaims.

With the inventive method of at least one embodiment for reconstructing image data of an object under examination from measured data, the measured data was detected beforehand during a relative rotational movement between a radiation source of a computer tomography system and the object under examination. A radiation scatter correction variable is determined, which is subjected to a low pass filtering. A connection of the filtered radiation scatter correction variable with the measured data takes place, and image data is reconstructed from the measured data corrected in this way.

The radiation scatter correction variable is used to eliminate or prevent unwanted influences, which the radiation scatter has on the images reconstructed from the measured data contaminated by radiation scatter. In the case of single-source devices, this relates to forward scatter, and in the case of dual-source devices, this relates to both forward scatter and also transverse scatter.

With the present method of at least one embodiment, the effects of the radiation scatter are not only eliminated or reduced until after the image reconstruction has taken place, but instead even before the image reconstruction. In other words, the radiation scatter correction variable acts directly on the measured data. This takes place by connecting the radiation scatter correction variable to the measured data. The connection corresponds to a mathematical operation, for which various possible embodiments exist.

The radiation scatter correction variable is first determined, then filtered and then connected to the measured data. This means that the filtering allows for an effect on the radiation scatter correction variable which does not relate to uncorrected measured data. The low pass filtering means that the low frequency information of the radiation scatter correction variable is retained and, accordingly, the high frequency information of the radiation scatter correction variable is eliminated. This enables the properties of the radiation scatter correction variable to be directly influenced.

The low pass filtering preferably relates to the spatial frequency, with the spatial frequency being the Fourier-transformed variable at the location. This location designates the positions on the detector; if a multiline detector is used, a matrix of measured values is present per projection angle, with each measured value belonging to a detector element with a specific local coordinate.

In one embodiment of the invention, the radiation scatter is measured in order to determine the radiation scatter correction variable. The radiation scatter correction variable does not have to correspond directly to these measured variables; it can also be obtained from the measurements by means of calculation. The radiation scatter can be measured particularly during the measured data acquisition.

It is also possible for radiation scatter to be calculated in order to determine the radiation scatter correction variable. This calculation can be performed in combination with a measurement of radiation scatter. In accordance with this embodiment, the radiation scatter is preferably not measured, but is exclusively determined by way of the calculation.

It is advantageous if a standardization and logarithmization of a measured or calculated radiation scatter intensity takes place in order to determine the radiation scatter correction variable. In this way, the radiation scatter correction variable is made available in the form in which intensity measured data usually enters the image reconstruction.

In accordance with one embodiment of the invention, the radiation scatter correction variable is determined per detector element. This means that the radiation scatter correction variable does not consist of a single value, but instead includes a plurality of values, with each value being assigned to a detector element. In particular, a value of the radiation scatter correction variable can be determined for each projection angle at which measured data was detected, for each detector element.

According to a development of at least one embodiment of the invention, the low pass filtering effects a smoothing of noises of the radiation scatter correction variable. If this noise is eliminated, measured data corrected by the radiation scatter is also less prone to noise, thereby increasing the quality of the images reconstructed herefrom.

According to one embodiment of the invention, the low pass filtering is implemented in the detector channel direction. The term channel direction is understood to mean the direction along a detector line; in this case movement is through the different detector elements of a line. The low pass filtering in the detector channel direction therefore combines values of the radiation scatter correction variable, which belong to different detector elements of a line, with one another.

Alternatively or in addition to filtering in the detector channel direction, the low pass filtering can be implemented in the detector line direction. This direction is at right angles to the channel direction. Movement is therefore from one detector element to the detector elements of the same channel position of the other lines. It is therefore possible to perform a one-dimensional filtering in the detector channel direction, or a one-dimensional filtering in the detector line direction, or a two-dimensional filtering in the channel and line direction.

The method is particularly suited to measured data which was detected during a dual-source CT measurement. The radiation scatter here represents a particularly large problem as a result of the transverse scatter.

The connection of the filtered radiation scatter correction variable with the measured data can be performed by an addition or subtraction for each respective detector element. In some instances, these computational operations can include a weighting of the radiation scatter correction variable and/or measured data.

The inventive control and computing unit of at least one embodiment is used to reconstruct image data of an object under examination from measured data of a CT system. It includes a program memory for storing program codes, with, if necessary inter alia, a program code being present herein, which is suited to implementing a method of the afore-described type. The inventive CT system of at least one embodiment includes such a control and computing unit. Furthermore, it can contain other components, which are needed to detect measured data for instance.

The inventive computer program of at least one embodiment has program codes and/or segments/modules, which are suited to implementing the method of the afore-described type if the computer program is executed on a computer.

The inventive computer program product of at least one embodiment includes program codes and/or segments/modules which are stored on a machine-readable data carrier, the program code segments/modules being suited to implementing the method of the afore-described type, if the computer program is run on a computer.

### BRIEF DESCRIPTION OF THE DRAWINGS

The invention is described in more detail below with reference to an example embodiment, in which;

FIG. 1: shows a first schematic representation of an example embodiment of a computer tomography system having an image reconstruction component,

FIG. 2: shows a second schematic representation of an example embodiment of a computer tomography system having an image reconstruction component,

FIG. 3: shows a dual-source CT data acquisition with transverse scatter,

FIG. 4: shows a flowchart.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

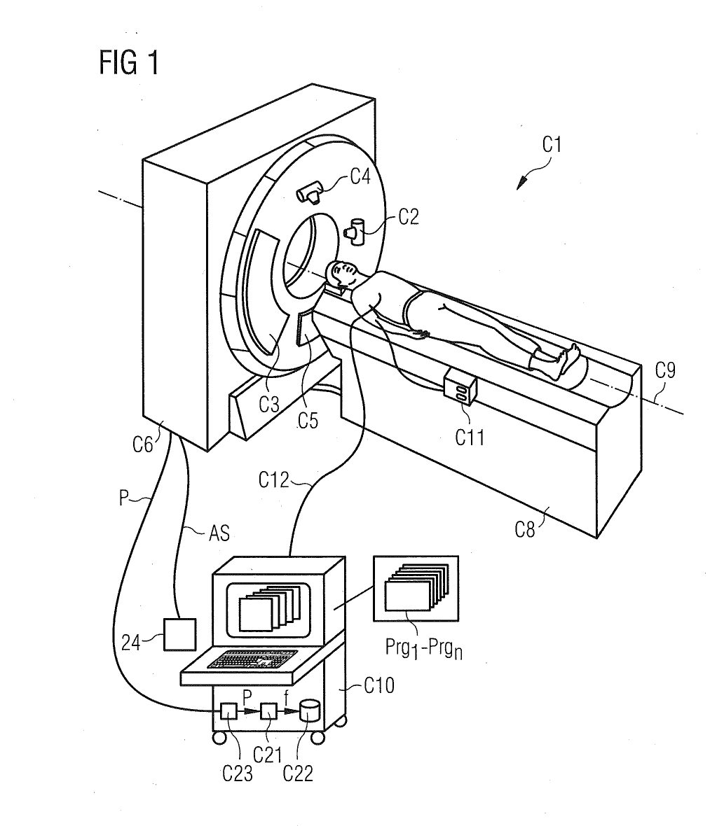

FIG. 1 firstly shows a schematic representation of a first computer tomography system C1 having an image reconstruction facility C21. A closed gantry (not shown here) is located in the gantry housing C6, on which closed gantry a first x-ray tube C2 with an opposite detector D3 is arranged. A second x-ray tube C4 with an opposite detector C5 is optionally arranged in the CT system shown here, so that a higher time resolution can be achieved by the emitter/detector combination also available, or “dual-energy” examinations can also be implemented during the use of different x-ray energy spectra in the emitter/detector systems.

The CT system C1 also has a patient couch C8, upon which a patient can be moved into the measuring field during the examination along a system axis C9, also known as z-axis, with it being possible for the scanning itself to take place both as a purely circular scan without advance of the patient exclusively in the examination region of interest. The x-ray source C2 and/or C4 rotates here about the patient. C3 and/or C5 run in parallel here relative to the x-ray source C2 and/or C4 of the detector, so as to detect projection measured data which is then used to reconstruct sectional images. Alternatively to a sequential scan, in which the patient is moved step by step between the individual scans through the examination field, the possibility of a spiral scan is naturally also available, in which the patient is moved continuously along the system axis C9 through the examination field between the x-ray tube C2 and/or C4 and detector C3 and/or C5 during the peripheral scanning with the x-ray radiation. As a result of the movement of the patient along the axis C9 and the simultaneous cycle of the x-ray source C2 and/or C4, a helical path is produced relative to the patient during the measurement in the case of a spiral scan for the x-ray source C2 and/or C4. This path can also be achieved by the gantry being moved along the axis C9 when the patient is not moved. It is also possible to continuously and periodically move the patient to and fro between two points.

The CT system 10 is controlled by a control and computing unit C10 having computer program codes Prg₁ to Prgₙ present in a memory. It should be pointed out that these computer program codes Prg₁ to Prgₙ are of course also able to be contained on an external storage medium and able to be loaded if necessary into the control and computing unit C10. Acquisition control signals AS can be transmitted from the control and computing unit C10 via a control interface 24 in order to activate the CT system C1 according to certain measuring protocols.

The projection measured data p acquired by the detector C3 and/or C5 (also referred to below as raw data) is transferred to the control and computing unit C10 by way of a raw data interface C23. This raw data p is then, if necessary after a suitable preprocessing, further processed in an image reconstruction component C21. The image reconstruction component C21 is realized in this exemplary embodiment in the control and computing unit C10 in the form of software on a processor, e.g. in the form of one or more of the computer program codes Prg₁ to Prgₙ. What has already been stated in respect of the control of the measuring process also applies to the image reconstruction, that the computer program codes Prg₁ to Prgₙ are also contained on an external storage medium and can if necessary be loaded into the control and computing unit C10.

The image data f reconstructed by the image reconstruction component C21 is then stored in a memory C22 of the control and computing unit C10 and/or is output onto the screen of the control and computing unit C10 in a conventional fashion. It can also be fed into a network connected to the computer tomography system C1, for instance a radiological information system (RIS), by way of an interface (not shown) in FIG. 1 and can be stored in a mass storage device which is accessible there or output as images.

The control and computing unit C10 may additionally also execute the function of an EKG, with a line C12 being used to obtain the EKG potentials between the patient and control and computing unit C10. In addition, the CT system C1 shown in FIG. 1 also has a contrast agent injector C11, by way of which additional contrast agent can be injected into the bloodstream of the patient, so that the vessels of the patient, in particular the heart chambers of the beating heart, can be better illustrated for instance. Furthermore, there is also the possibility here of implementing perfusion measurements, for which the proposed method is likewise suited.

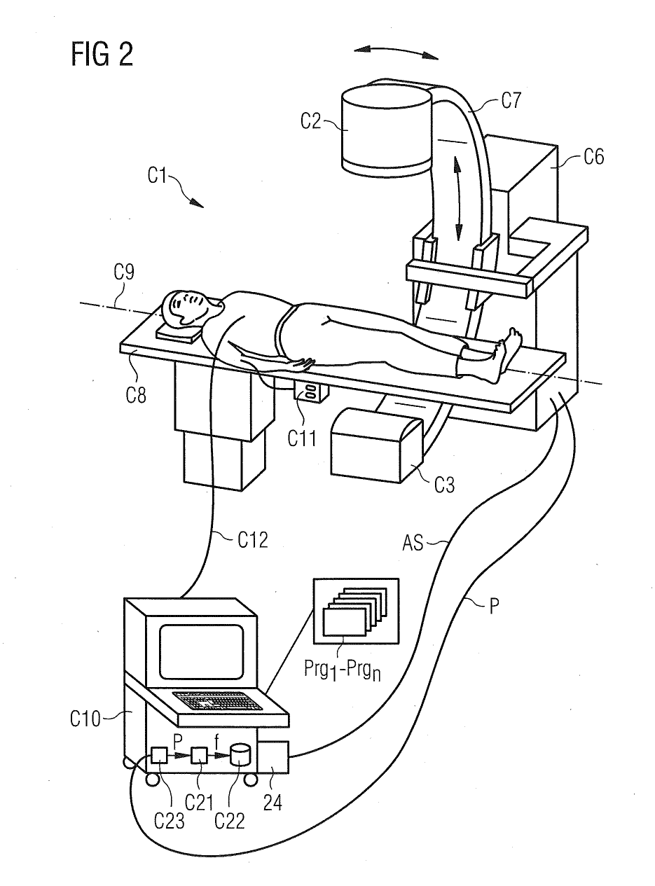

FIG. 2 shows a C-arm system, in which, contrary to the CT system in FIG. 1, the housing C6 supports the C-arm C7, to which the x-ray tube C2 on the one hand and the opposite detector C3 on the other hand are fastened. The C-arm C7 is likewise pivoted about a system axis C9 for a scan, so that a scan can take place from a plurality of scanning angles and corresponding projection data p can be determined from a plurality of projection angles. Similarly to the CT system from FIG. 1, the C-arm system C1 in FIG. 2 also has a control and computing unit C10 of the type described in FIG. 1.

An embodiment of the invention can be used in both of the systems shown in FIGS. 1 and 2. Furthermore, it can basically also be used for other CT systems, e.g. for CT systems having a detector forming a complete ring.

For CT devices with detectors extended in the longitudinal direction of the patient, i.e. in the z-direction, the radiation scatter limits the image quality as a result of the forward scatter. Forward scatter means that an x-ray quantum is not absorbed in the object under examination, but is instead scattered while changing direction and reaches the detector belonging to the x-ray source. This is disadvantageous since the x-ray quantum is “thrown off course” by way of the scatter and is thus measured in the incorrect detector element. Only those x-ray quanta which reach the respective detector element from the x-ray source according to a straight beam are desired for the image reconstruction. Accordingly, an x-ray quantum, which has not moved on a straight path of this type, since its direction was changed as a result of scatter, carries incorrect information for the image reconstruction.

The forward scatter increases approximately linearly with the z-coverage of the detector. This is because the probability of an x-ray quantum being scattered in the object under examination increases with an increasing width of the scanned layer, which corresponds to the z-coverage of the detector.

The radiation scatter produces artifacts in the images. In particular, dark zones, wide, dark lines and cupping effects, i.e. indentations or bulges, are visible in the reconstructed images. Therefore the radiation scatter does not bring about a uniform deterioration across the overall image. The reason for this is that the scatter does not take place uniformly, but instead as a function of the weakening of the tissue; the more a tissue absorbs the x-ray radiation, the more it also scatters the same. It is also impairs the signal-to-noise ratio of the images, so that a higher radiation dose has to be used in order to achieve a desired signal-to-noise ratio.

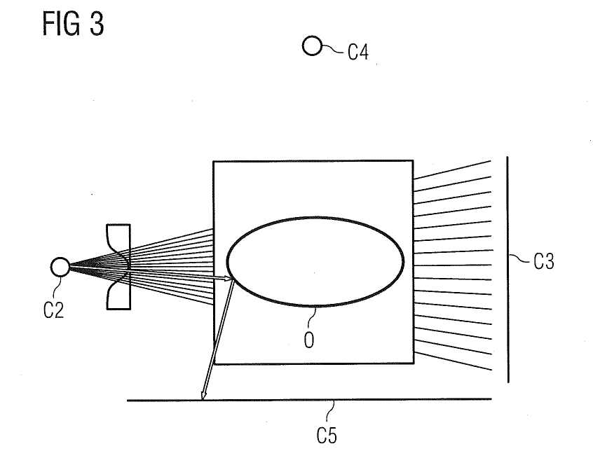

The transverse scatter is still added to the forward scatter for dual-source CT devices, which is illustrated with the aid of FIG. 3. The representation in FIG. 3 shows a section through the recording geometry at right angles to the z-axis. The two x-ray sources C2 and C4 can be seen, as can the opposite detectors C3 and C5. The detectors are each shown as a line. This line corresponds to a detector line, which comprises a plurality of detector elements and/or pixels. Further detector lines may be present adjacent in the z-direction and thus not visible in the representation.

The radiation of the x-ray source C2 penetrates the object under examination O and reaches the detector C3, and the radiation of the x-ray source C4 penetrates the object under examination O and reaches the detector C5. The transverse scatter strikes the surface of the object under examination O in particular. A beam which strikes the surface of the object under examination O from the x-ray source C2 and is scattered almost at right angles therefrom is marked by the thick arrow. This transverse scatter is detected by the detector C5, which is actually used to measure the radiation of the x-ray source C4.

In respect of radiation scatter, dual-source CT devices behave approximately like single-source CT devices having a detector which is twice as wide in the z-direction. The radiation scatter finally limits the maximum possible z-coverage of the detector in a CT device.

In the prior art, collimators are used on the detector side to reduce the radiation scatter. These are sheets which are attached in front of the detector and are used to only allow x-rays to pass from a certain direction relative to the respective detector element. With an increasing z-coverage of the detector and thus increasing radiation scatter intensity, the grid ratio of the collimators, in other words the ratio of the height of the sheet to the width of the detector element, has to be enlarged for the same efficiency, thereby meeting striking technological limits. The mechanical stability of the collimator sheets is particularly problematical here, since these are not permitted to oscillate even with the highest rotational frequencies. The use of grid-type collimators, which collimate both in the image plane and also in the z-direction, offers a better radiation scatter suppression, but is however extremely complicated and expensive. Collimators are on the whole restricted in terms of their efficiency, are technically complicated and expensive. By themselves, they are not able to solve the radiation scatter problem in the case of single-source CT devices with a detector which is expanded in the x-direction and in particular in the case of dual-source CT devices. With dual-source CT devices, there is an additional problem that, in respect of single-source CT devices, in the case of a transverse-scattered x-ray quantum, the direction, with which the x-ray quantum strikes the incorrect detector, may be the correct one so that it cannot be stopped by the collimator.

Computational scatter corrections are possible as further methods of reducing the radiation scatter. The radiation scatter signal is initially determined here for each detector element. This can either take place by means of a direct measurement, by additional detector elements being attached, if necessary, to both sides of the detector, e.g. in the z-direction outside of the detector. This procedure is also suited to a multiline detector, since the radiation scatter changes very little in the z-direction. Alternatively, the radiation scatter beam can be determined by model assumptions; calculations are performed here to determine how the radiation scatter is to be seen in the case of certain object forms.

The specific radiation scatter signal is then wholly or partially deducted from the measuring signal during the data acquisition or image reconstruction. In particular, if the radiation scatter signal is measured directly during the examination, methods of this type for scatter correction are very effective for artifact suppression. Nevertheless they have a decisive disadvantage: the average values of the measuring signal are corrected in this way by the radiation scatter so that these average values actually correspond to the average measured values present without radiation scatter. This deduction of the radiation scatter nevertheless has an advantageous effect on the average measured values, but not however on the noise. Since, despite correction, the quantum noise of the radiation scatter remains in the signal obtained after the correction, the additional quantum noise introduced by the radiation scatter cannot be subtracted.

All algorithmic methods for scatter correction can therefore significantly reduce the artifacts (darkenings, indentations etc.) caused by the radiation scatter, however always at the price of an increased image noise. To maintain a desired signal-to-noise ratio, a higher radiation dose is therefore needed for the object under examination than in the case of a CT device with a detector which is extended only slightly in the z-direction. Engel et al (Medical Physics 2008, 35(1):318-332) report that in order to maintain the signal-to-noise ratio for a standard thorax phantom in the case of a single-source CT device with 16 cm z-coverage in the rotational center when using a computation scatter correction compared with a single-source CT device with 2 cm z-coverage, the radiation dose has to be increased by 54%.

With a dual-source CT device having 4 cm z-coverage, a 20% larger dose is needed in comparison with a single-source CT device having a 2 cm z-coverage. A notional dual-source CT device with for instance an 8 cm z-cover for both detectors would render necessary a dose increase by 47% in the case of a standard thorax examination. The situation becomes even more dramatic if a relatively low-attenuation standard thorax is not taken into consideration, but instead a CT scan in the abdomen area, particularly in the case of obese patients.

The knowledge underlying the procedure described below is that the radiation scatter-corrected, logarithmized CT raw data, which represents the input data for the image reconstruction, can be split by suitable mathematical deformations into the measured logarithmized raw data and logarithmized correction data. A suitable low pass filtering is then applied to the logarithmized correction data in order to reduce the noise.

Let Iₜ^{k}=Iₚ^{k}+Iₛ^{k} be the intensity measured in a detector channel k after x-raying the object under examination. Iₚ^{k} is the ideal attenuated intensity, i.e. the measuring result, which has to materialize without radiation scatter. Iₛ^{k} is the scatter component present in the detector element k. This includes direct scatter, i.e. forward scatter, and in the case of dual-source CT devices the transverse scatter. Iₛ^{k} is measured as described above or calculated by means of model assumptions. For radiation scatter correction, Iₛ^{k} is subtracted from the measured intensity Iₜ^{k}, in order to obtain the desired ideal attenuated intensity Iₚ^{k}=Iₜ^{k}−Iₛ^{k}.

Input data for the CT image reconstruction is the logarithmized values fₚ^{k}=−ln(Iₚ^{k}/I₀) where I₀ is a standardization intensity, namely the intensity of the unattenuated x-ray beam.

whereby

$$\begin{matrix}{f}_{p}^{k} = - ln ( {I}_{p}^{k} / {I}_{0} ) \\ = - ln ( \frac{{I}_{t}^{k} - {I}_{s}^{k}}{{I}_{0}} ) \\ = - ln ( {I}_{t}^{k} / {I}_{0} ) - ln ( 1 - {I}_{s}^{k} / {I}_{t}^{k} ) \\ = {f}_{t}^{k} + {f}_{s}^{k}\end{matrix}$$

applies.
fₜ^{k}=−ln(Iₜ^{k}/I₀) is the raw data measured, logarithmized and standardized in the detector channel k including radiation scatter. fₛ^{k=-ln(}1−Iₛ^{k}/Iₜ^{k}) is logarithmized and standardized “correction data”, which represent the channel-dependent scatter correction.

The ideal logarithmized data fₚ^{k} desired for the image reconstruction therefore results in accordance with the above deformation by adding the measured, logarithmized raw data fₜ^{k}=−ln(Iₜ^{k}/I/₀) including radiation scatter, and the correction data, fₛ^{k}=−ln(1−Iₛ^{k}/Iₜ^{k}), which remove the scatter component. Nevertheless, the correction data fₛ^{k} not only corrects the radiation scatter but instead introduces additional high-frequency noise in the image, namely essentially by the necessary division of the scatter beam intensity Iₛ^{k} estimated in a model-type fashion or by means of additional measurements and the caused measured intensity Iₜ^{k}.

The image artifacts produced by radiation scatter, such as darkenings, wide lines, indentations etc. are spatially low-frequency and contain practically no high-frequency components. In comparison, the image noise caused by the scatter correction is in the high-frequency range. If information is located in the spatially low frequency range, this means that it contains no fine details. By contrast, high local frequencies indicate finely-structured information, like for instance small objects, sharp edges or fine-grain noise. The local frequency is herewith the Fourier-transformed variable at the location. This variable can be viewed as lines per centimeter which are available in order to represent the object imaged in the spatial area.

It is therefore desirable to retain the information administered by the scatter correction in the low frequency part, since these are used to eliminate the low frequency image artifacts. On the other hand, the scatter correction in the high-frequency part introduces unwanted information, namely the noise which one would like to rule out.

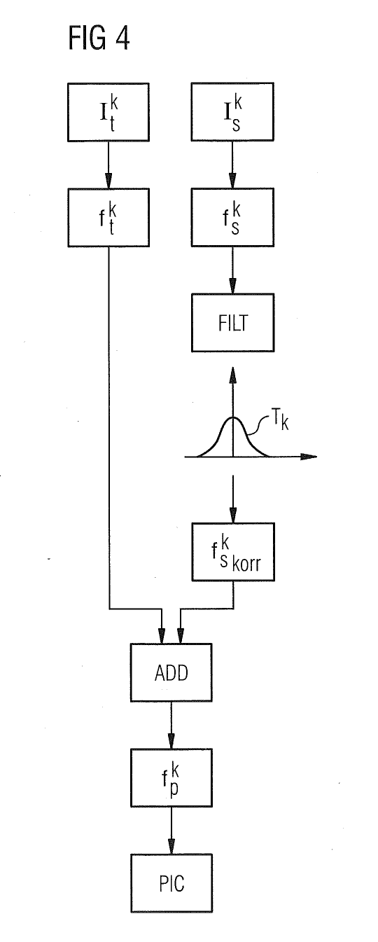

FIG. 4 shows a flowchart of a method for image reconstruction. This can be applied both to single-source and also dual-source recordings. The measured data Iₜ^{k} is firstly detected. The radiation scatter Iₛ^{k} is determined in a temporally offset or simultaneous fashion. fₛ^{k} is calculated from the measured data Iₜ^{k} in the manner described above by means of standardization and logarithmization fₜ^{k} and from Iₛ^{k}.

Before adding fₜ^{k} and fₛ^{k} in step KORR, a low pass filter T^{k} is applied to the correction data fₛ^{k} in step FILT in order to obtain filtered correction data f_{s korr}^{k}. The filtering is implemented in the channel direction, i.e. the individual measuring results of the detector elements of a detector line are connected to one another by way of the filter operation. If the filtering takes place in the local space, this is a convolution. This calculation can alternatively be implemented as multiplications in the frequency space. To this end, the data fₛ^{k} is previously Fourier-transformed and processed with the convolution core in the frequency domains, i.e. the similarly Fourier-transformed convolution function. The calculation takes place in the frequency space according to

$${Tf}_{s}^{k} = {\sum }_{m} {T}^{k - m} {f}_{s}^{m} ,$$

i.e. filtered correction data Tfₛ^{k}=f_{s korr}^{k} is calculated channel by channel.

The illustration of the filter T^{k} in FIG. 4 corresponds to the representation in the frequency space. The filter function T^{k} is represented in the local space by a Fourier transformation being implemented.

The low pass filter T^{k} is constructed such that its Fourier-transform has the value 1 with the frequency zero. Below a selectable limit frequency, the Fourier transform remains close to 1, above this selectable limit frequency, it is zero or practically zero.

The low pass filtering FILT means that the noise component of fₛ^{k} is reduced. With a suitably selected limit frequency, the low frequency correction components remain in the filtered correction term f_{s korr}^{k}, while the high frequency noise is suppressed. The selection of the limit frequency essentially depends on the detector geometry.

In step ADD, the total fₜ^{k}+f_{s korr}^{k} is formed from the measured, logarithmized raw data fₜ^{k} and the correction data f_{s korr}^{k} filtered channel by channel. The result, fₚ^{k}, is the input data for a standard CT image reconstruction. The CT image PIC is determined from fₚ^{k} by means of an image reconstruction algorithm.

Alternatively, the filtering can take place in the line direction (z direction) instead of in the channel direction k. Furthermore, the filtering can also take place in a multi-dimensional fashion, e.g. two-dimensionally both in the channel direction k and also in the line direction (z-direction).

A further possibility is to specify the correction term fₛ^{k} not exactly as fₛ^{k}=−ln(1−Iₛ^{k}/Iₜ^{k}), but instead to approximate the same by means of a Taylor development. This may be advantageous since the 1n calculation is complicated, so that computing time can be saved.

The method proposed here enables the reduction of typical scatter beam artifact (dark zones, wide, dark lines, indentation-type darkenings etc) by means of a computational correction even with high scatter beam intensities. Contrary to conventional scatter correction methods, the image noise is however not increased significantly compared with the uncorrected image, as a result of which the dose efficiency of the CT device compared with the conventional scatter correction method is significantly improved. It is particularly advantageous here that, after calculating the filtered correction term f_{s korr}^{k}, no additional outlay is produced compared with conventional scatter correction methods. The scatter correction can namely take place in a favorable fashion by filtering raw data using a subsequently simpler image reconstruction.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combineable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, computer readable medium and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable medium and is adapted to perform any one of the aforementioned methods when run on a computer device (a device including a processor). Thus, the storage medium or computer readable medium, is adapted to store information and is adapted to interact with a data processing facility or computer device to execute the program of any of the above mentioned embodiments and/or to perform the method of any of the above mentioned embodiments.

The computer readable medium or storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

The invention was described above with reference to an exemplary embodiment. It goes without saying that numerous changes and modifications are possible without departing from the scope of the invention.

What is claimed is:

1. A method for reconstructing image data of an object under examination from measured data, the measured data including data detected during a relative rotational movement between a radiation source of a computer tomography system and an object under examination, the method comprising:
determining a radiation scatter correction variable;
subjecting the radiation scatter correction variable to a low pass filtering;
correcting measured data using the filtered radiation scatter correction variable; and
reconstructing the image data from the corrected measured data.

2. The method as claimed in claim 1, wherein radiation scatter is measured to determine the radiation scatter correction variable.

3. The method as claimed in claim 1, wherein radiation scatter is calculated to determine the radiation scatter correction variable.

4. The method as claimed in claim 1, wherein a standardization and logarthimization of a measured or calculated radiation scatter intensity takes place to determine the radiation scatter correction variable.

5. The method as claimed in claim 1, wherein the radiation scatter correction variable is determined per detector element.

6. The method as claimed in claim 1, wherein the low pass filtering effects a smoothing of noise of the radiation scatter correction variable.

7. The method as claimed in claim 1, wherein the low pass filtering is implemented in the detector channel direction.

8. The method as claimed in claim 1, wherein the low pass filtering is implemented in the detector line direction.

9. The method as claimed in claim 1, wherein the measured data includes data detected during a dual-source measurement.

10. The method as claimed in claim 5, wherein the correction of the measured data with the filtered radiation scatter correction variable takes place by addition or subtraction for each respective detector element.

11. A control and computing unit for reconstructing image data of an object under examination from measured data of a CT system, comprising:
a program memory for storing program codes to, when executed by the control and computing unit,
determine a radiation scatter correction variable;
subject the radiation scatter correction variable to a low pass filtering;
correct measured data using the filtered radiation scatter correction variable; and
reconstruct the image data from the corrected measured data.

12. A CT system comprising the control and computing unit as claimed in claim 11.

13. A computer program, stored on a non-transitory computer readable medium, comprising program code segments to implement the method as claimed in claim 1, upon the computer program being executed on a computer.

14. A computer program product, comprising program code segments of a computer program which are stored on a non-transitory machine-readable data carrier, to implement the method as claimed in claim 1, upon the computer program being executed on a computer.

15. The method as claimed in claim 2, wherein a standardization and logarthimization of the measured radiation scatter intensity takes place to determine the radiation scatter correction variable.

16. The method as claimed in claim 3, wherein a standardization and logarthimization of the calculated radiation scatter intensity takes place to determine the radiation scatter correction variable.

17. The method as claimed in claim 6, wherein the low pass filtering is implemented in the detector channel direction.

18. The method as claimed in claim 6, wherein the low pass filtering is implemented in the detector line direction.

19. A non-transitory computer readable medium including program segments for, when executed on a computer device, causing the computer device to implement the method of claim 1.
